(19) United States
(12) Patent Application Publication
(10) Pub. No.: US 2024/0206950 A1
(43) Pub. Date: Jun. 27, 2024

(54) METHOD FOR OPERATING AN ELECTRO SURGICAL GENERATOR

(71) Applicant: OLYMPUS WINTER & IBE GMBH, Hamburg (DE)

(72) Inventors: Jens KRÜGER, Zeuthen (DE); Fabian STOPP, Berlin (DE); Anne KWIK, Berlin (DE)

(73) Assignee: OLYMPUS WINTER & IBE GMBH, Hamburg (DE)

(21) Appl. No.: 18/392,425

(22) Filed: Dec. 21, 2023

Related U.S. Application Data
(60) Provisional application No. 63/435,095, filed on Dec. 23, 2022.

(51) Int. Cl. A61B 18/12

(52) U.S. Cl. CPC A61B 18/1206

(57) ABSTRACT

A method for controlling an electro surgical generator for controlling electro surgical instruments connected to the electro surgical generator, including a plurality of interconnected regular modules, the plurality of interconnected regular modules including at least one socket module for connecting the electro surgical instrument, and at least one first inverter module for generating a feed signal for providing a high frequency energy for at least one electro surgical instrument connected to the socket module, wherein, each regular module communicates at least with another regular module and/or with a communication module, each regular module is sending a communication frame at an individual repetition frequency and the individual repetition frequency is varied depending on a state of the electro surgical generator.

The present invention is directed to a method for operating an electro surgical generator. The invention is also directed to such electro surgical generator.

Electro surgical generators control electro surgical instruments connected to such generator. In particular there is at least one inverter for generating a high frequency feed signal in order to feed an electro surgical instrument connected to the electro surgical generator.

For safety applications in such generators the generators should ensure that components are able to react on an input, such as measurements, with a correct output, in particular the control of the feed signal, i.e. the control of provided high frequency energy, in a timely manner. To enable this, components such as an inverter module can be tested for reactivity. If a component is stuck or has a malfunction, this can be detected via a frequent test routine. In such frequent test routine for each relevant component it is checked whether it is still operating properly, at least if it is still sending confirmation signals on a frequently basis. Such frequent test can be called a heartbeat system as it frequently checks whether the particular component is still in operation.

Such reaction time on failures can be defined as failure tolerance time (FTT).

In a worst case scenario the generator outputs a maximum HF energy power which is not controllable. In the tissue of the patient this energy generates heat and burns the tissue, resulting in carbonization. The failure tolerance time takes this failure scenario and calculates a time after which the tissue is damaged irreversibly. In a failure scenario the safety system must be able to shut down the generator in this timeframe. As an example for a generator which can output 2500 W in a single fault condition, the failure tolerance time can be calculated to be about 200 ms or in one example 189 ms.

In a heartbeat system the supervision time should be well below the failure tolerance time to ensure that a malfunctioning subcomponent is detected and there is enough time left to shut down the HF energy output. However, this creates a high amount of communication traffic because every node which can be representative of a component or module connected to a communication network, has to send data at least one or more times in this timeframe. Accordingly, each connected component, in particular module sends data at least every 200 ms according to the example given above. That is a minimum and if it sends two times in said timeframe, it sends data every 100 ms. Not only one module but all modules connected to said network may thus send within that timeframe, resulting in a lot of communication traffic.

In a distributed communication system, such as a system with nodes connected to a CAN, this can lead to high bus load for this communication.

According to one prior art a control software of a generator may be distributed on various modules. A central module may send a signal, which can be called a heartbeat signal, to all other modules which need to answer within a predetermined time. Otherwise, the generator changes over into an error state.

Accordingly, the object of the present invention is to provide a solution to at least one of the above explained problems. In particular, a solution shall be suggested that entrusts a high safety level but at the same time avoiding an overload of a communication system in particular a communication network. At least an alternative solution with respect to known solutions shall be suggested.

According to the invention a method according to claim 1 is suggested. Accordingly a method for operating an electro surgical generator is suggested. Such electro surgical generator is adapted for controlling electro surgical instruments connected to the electro surgical generator. In particular the generator provides HF energy to at least one electro surgical instrument. Further control tasks are executed by such generator such as receiving and forwarding inputs from a user and displaying states of the generator.

The electro surgical generator used comprises a plurality of interconnected regular modules comprising at least one socket module for connecting an electro surgical instrument. Such socket module may also be denominated as output module. The electro surgical instrument may be plugged into such socket module. The socket module may comprise a sensor indicating whether and possibly what kind of electro surgical instrument is connected. The socket module may also have a sensor checking an amplitude of a feed signal. The socket module may also have an information connection receiving information on an operating state of the connected electro surgical instrument. Any such information received can be communicated inside the electro surgical generator to other modules in the generator and/or to a communication module. There may be a communication between the socket module and an inverter module which provides the feed signal to the socket module and thus to the connected electro surgical instrument.

The regular modules also comprise at least one first inverter module for generating a feed signal for providing a high frequency energy for at least one electro surgical instrument connected to the socket module. Such inverter module may thus generate, by using a pulse width modulation, a voltage signal having a particular frequency and resulting in a corresponding current signal delivered to the socket module and thus to a connected electro surgical instrument. The high frequency energy is thus energy provided by a feed signal having a frequency in the range of 20 kHz to 500 KHz.

It is further suggested that each regular module communicates at least with another regular module or with a communication module. Accordingly, all these regular modules are at least adapted for communication. The regular modules may communicate directly with each other and/or via the communication module. Accordingly, a centralized as well as a decentralized communication, i.e. network architecture can be possible. It is suggested that an inverter module communicates with a socket module. The socket module may receive from a lever or switch from the connected electro surgical instrument a request for providing a feed energy or a request for stopping to supply the feed energy. Accordingly, this information is communicated with the corresponding inverter module. The inverter module is accordingly generating the wanted high frequency feed signal. In the same manner a request for stopping the feeding can be received by the socket module and communicated to the inverter module.

It is further suggested that each regular module communicates at least with another regular module and/or with a communication module. For this purpose of communication each of the regular modules and/or communication module may have a node for participating in the communication and in particular for participating in a network system such as a network bus system. By means of this communication the operation of the regular modules can be controlled. For this purpose each of the regular modules may have its own control module and accordingly each regular module may be provided as an intelligent module that can receive information, send information and control its own operation based on such information.

It is further suggested that each regular module is sending a communication frame at an individual repetition frequency. Such individual repetition frequency is a frequency indicating how often the particular regular module is sending said communication frame, e.g. that can be at 2 Hz or at 12 Hz, to just give one example for variation. All regular modules have such individual repetition frequency but this individual repetition frequency may be the same for all regular modules.

It is further suggested that the individual repetition frequency is varied depending on a state of the electro surgical generator. Such state of the electro surgical generator can be that the electro surgical generator is in operation or in standby. It is particularly suggested that the individual repetition frequency is smaller and thus the communication frame is sent less often, when the electro surgical generator is in standby. On the other hand, it is suggested that the individual repetition frequency is higher if the electro surgical generator is in operation. That may in particular mean that at least the first inverter module generates a feed signal and is thus providing said high frequency energy to an electro surgical instrument.

This change of the individual repetition frequency depending on the state of the electro surgical generator may also be the same for all regular modules and thus for all individual repetition frequencies. The term “individual repetition frequency” is particularly used to indicate that each regular module is individually controlled, i.e. is controlling itself individually. Therefore, each regular module has its own individual repetition frequency but according to at least one embodiment it is suggested that the individual frequencies are the same for all regular modules. And accordingly, if the individual repetition frequency is raised depending on the state of the electro surgical generator, all individual repetition frequencies may be raised. On the other hand, if depending on the state of the electro surgical generator the individual repetition frequency is lowered, all individual repetition frequencies may be lowered.

However, the individual repetition frequencies may be different between different regular modules.

Accordingly, with respect to a known system explained above, the generator is modified such that the high failure tolerance time related heartbeat frame frequency can be altered with a lower frequency if the generator is not in an activation state. It was found that failures that occur without contact to the patient and without any HF output have nearly no risk involved.

It was thus found that so far every heartbeat was sent in a fixed time frame. This time frame is specified on the basis of a failure tolerance time (FTT) calculation, i.e. based on a maximum time allowed HF activation in which no patient harm is caused. These FTT values are often very small to react as quickly as possible, for example within 140 ms. However, every heartbeat message creates system and communication load. When considering distributed communication nodes and an interconnecting bus, the heartbeat messages will infer with other communication and contribute to a high bus- or communication load.

Accordingly, it was realized that as the FTT is calculated on tissue effect on a given HF energy and application time, this approach is only valid during actual HF activation. Most of the time, the generator is in a standby like mode, where each component does self-checks and supervision of other components. In this time, a relaxed timing can be used without reducing the safety of the device.

Accordingly, it is suggested a supervision concept which has more than one operating mode. I.e. during standby of the device, each component signals its heartbeat with much less repetition rate, i.e. lower heartbeat frequency and thus lower repetition frequency. Therefore, the communication bus load or the communication system load can be reduced significantly. During activation, the central control node sends out an activation status, which is registered by all components, i.e. by all regular modules. During this time, the heartbeat frequency is increased to a value, where the FTT for HF activation can be met.

Furthermore, it was found that this approach can be extended with a flexible heartbeat frequency, i.e. with a flexible repetition frequency. Each module can alter its heartbeat frequency based on an inner state. For example, the socket module can reduce the heartbeat frequency to a lower value, when no instrument is connected. As all modules communicate their state in the heartbeat-datagram, the supervisor application can check if heartbeat frequency and state matches to each other and can reliably take actions when a module is running out of specification. Accordingly, this can be done by a communication module which can also be denoted as the supervisor application.

According to one aspect the electro surgical generator further comprises the communication module for controlling a communication with the regular modules and/or each regular module frequently sends a communication frame at its individual repetition frequency to the communication module and/or to another regular module. As the communication module may have a supervisory function it can also be denominated as supervisory communication module.

Accordingly, the communication module is used for controlling the communication within the electro surgical generator. This way a centralized controller of the communication can be established and the communication module can act as one supervising node whereas one or more regular modules are connected as regular nodes to the supervising node. The connection point or connection interface of the communication module to the communication network in particular to the bus system can thus be denominated as the supervising node. Similar the connecting point or interface of a regular module to the communication network in particular to the bus system can be denominated as a regular node.

Preferably each node or module can send a CAN-frame which has at least two information, namely the information of the own node ID of the corresponding node and as a second information an inner state of operation. Such inner state of operation can for example be a boot-up, an error information, the information that the module is in standby, or the information that the module is busy, to give just some examples.

It is possible that additional information like error codes or detailed error information can be part of the CAN-frame and will thus be sent by each node.

Also during operation of the generator, each node regularly sends a heartbeat frame to the supervising node. Such heartbeat frame is thus a frame that is sent frequently with the repetition frequency. The frame sent may just comprise an information indicating that the particular module sending the frame is not having an error. The frequency in which these frames are sent, depends on the current state of the generator, in particular if the generator as such is in standby or in operation or in another mode, or it may depend on the state of the particular modules according to the possible inner states of operation mentioned above.

This way a high repetition frequency is possible for any safety relevant operation of the generator or of particular modules. For all other modules and/or situations, a lower repetition frequency is used and thus that enables to reduce a load on the communication system, in particular on the bus system.

In addition or alternatively, the electro surgical generator and/or each regular module perform a self-check during standby, wherein each regular module sends a communication frame at its individual repetition frequency. Accordingly, it is suggested that during standby there is still performed a regular self-check. This way it can be ensured that the generator is ready whenever there is a request of use. However, it is suggested that for such self-check during standby, the repetition frequency is lower, compared to a situation without standby, as no safety relevant operation is going on during standby.

According to one aspect, at least during a standby mode the individual repetition frequencies of two, more than two or all of the other regular modules are the same. This is suggested as it was found that changing the repetition frequency depending on the state of the generator is not only relevant for one regular module but for more regular modules and possibly even for all regular modules. By using the same repetition frequencies for two, more than two or all regular modules, the overall control of the generator can be simplified. It avoids to check for each single regular whether a high repetition frequency is necessary or not. Accordingly, mistakes are avoided by using the same repetition frequencies.

According to one aspect, some or all individual repetition frequencies are selected depending on an overall state of the electro surgical generator. Thus, if the electro surgical generator as a whole is in standby, the individual repetition frequencies can be selected accordingly. If, however, the electro surgical generator is in operation, such that at least one inverter module feeds an HF feed signal, a different individual repetition frequency can be chosen for all regular modules. In particular, this will be higher than at standby.

An overall state is thus defined not only based on a single regular module, but based on the overall situation of the electro surgical generator. The overall state of the electro surgical generator can be defined such that it is in standby, if everything of the electro surgical generator is in standby. It can be defined to be in operation if just one regular module of the electro surgical generator is in operation. This avoids mistakes as there is no need to distinguish which regular module is in operation. If just one is in operation, then the overall state is in operation and in particular all individual repetition frequencies are then put to a higher value.

However, it is also possible to distinguish between particular regular modules. Accordingly, one aspect is that one, some or all of the individual repetition frequencies are selected depending on a state of the corresponding regular module. Such individualization has the advantage that an overload of the communication system or bus system is avoided if only some regular modules are in operation. This way, only some individual repetition frequencies need to be high.

According to one aspect, it is suggested that during standby of the electro surgical generator some or all individual repetition frequencies are selected to be lower when compared to the electro surgical generator not being in standby. As explained above, in this way there is only a high load on the communication system or bus system if the electro surgical generator is not in standby. During standby, there is only a small load on the communication system or bus system that enables the communication system or bus system to provide other services such as updating and/or configuring the generator or some of the regular modules of the generator.

In particular, it is suggested that an individual repetition frequency is selected depending on an activation frequency being an inverse value of an activation time of an inverter module, wherein the activation time defines a time the inverter module needs to stop its feed signal. It is further suggested that during standby of the electro surgical generator an individual repetition frequency is selected being smaller than the activation frequency, in particular, at most half the size of the activation frequency. It is also suggested that when the electro surgical generator is not in standby, an individual repetition frequency is selected being larger than the activation frequency, in particular, at least double the size of the activation frequency.

Accordingly, it was found that a time the inverter module needs to stop its feed signal is an important time in view of safety. Such activation time is in particular directed to activating a stop. In other words, it could also be understood as a deactivation time. It was found that for safety reasons this activation time is an important value. It is usually short enough to avoid any harmful treatment on a patient. Based on that, it was found that such activation time should not be unduly extended by using a low repetition frequency. Accordingly, the repetition frequency should be that high that any information such as an order for stopping the feed signal should not take longer than the activation of a stop of feeding. Accordingly, it is suggested selecting a larger repetition frequency than the activation frequency. However, during standby that is not necessary and accordingly it is suggested that during standby the repetition frequency is smaller than the activation frequency. Being at least half the size of the repetition frequency makes it significantly lower at standby than without standby, resulting in a noticeable reduction of traffic on the communication network or communication bus.

In particular, it is suggested that the repetition frequency during operation, i.e. if there is no standby, is at least double the size of the activation frequency and this way it is ensured that the repetition frequency is significantly higher than the activation frequency and thus the main delay in case of an emergency shutdown depends on the ability of the particular regular module, i.e. the inverter module, and not on the communication time.

According to one aspect, during activation of the electro surgical generator as to leave a standby mode, the communication module sends a command to one, some or all of the regular modules to increase their individual repetition frequency and/or when the electro surgical generator enters the standby mode, the communication module sends a command to one, some or all of the regular modules to increase their individual repetition frequency.

Accordingly, such increasing or decreasing of the repetition frequency is controlled by the communication module and thus controlled in a centralized manner. This has the advantage that the communication module may receive all relevant information from all or most modules or components of the electro surgical generator. This way, the communication module also receives information on activation of the electro surgical generator, i.e. ending the standby mode, and also on entering the standby mode.

In particular, activating the electro surgical generator and thus leaving the standby mode can be time critical, as right after activation a failure or other malfunction may occur and in that case it is important that the repetition frequency is already high. This way any malfunction can be identified and communicated fast enough.

According to one aspect, it is suggested that one, some or all of the regular modules each individually alter their individual repetition frequency depending on their module state. In particular, the socket module decreases its individual repetition frequency, when an electro surgical instrument is retracted from the socket module. And it increases its individual repetition frequency when an electro surgical instrument is plugged into the socket module.

This way, there is a clear situation defined according to which the regular module can decrease or increase its own repetition frequency. In particular, increasing its individual repetition frequency can be important for safety reasons. As explained, once the electro surgical instrument is plugged into the socket module, the repetition frequency is immediately increased and thus with plugging in this electro surgical instrument there can immediately be a fast enough monitoring of this socket module and thus immediately the corresponding safety function can be ensured.

According to one aspect, the communication module frequently checks the individual repetition frequency of the regular modules depending on an individual module state of the corresponding module and/or depending on an overall state of the electro surgical generator and/or based on a given data base which is particularly stored in a memory of the communication module, and wherein the communication module provides an error signal and optionally initiates safety actions if at least one of the checked individual repetition frequencies deviates from the corresponding expected individual repetition frequency.

Accordingly, the communication module checks whether each regular module is not showing any error by frequently checking the individual repetition frequencies. This means that each regular module frequently sends a signal, in particular an okay signal and/or a data frame with the repetition frequency. Accordingly, if the individual repetition frequency is not met by one regular module, this is identified by the communication module which in turn provides the error signal.

However, the individual repetition frequency which the communication module is taking as a reference, i.e. the expected individual repetition frequency, may change. It changes depending on a module state and/or depending on an overall state of the electro surgical generator. Accordingly, depending on the module state and/or the overall state, the communication module should be expecting a particular individual repetition frequency. This expectation is thus based on the particular individual module state and/or based on the overall state of the electro surgical generator. This way, the check function provided by the communication module can be based on the individual module state and/or on the overall state of the electro surgical generator.

One way of doing this is to use a data base which can be a lookup table. In this data base or lookup table there are individual repetition frequencies assigned to particular states, i.e. to individual module states and/or to overall states of the electro surgical generator. Accordingly, the communication module checks the actual state. Based on this actual state, the data base or lookup table is used to find the corresponding repetition frequency. The thus found corresponding repetition frequency is then used for checking whether the particular regular module is sending data frames with the individual repetition frequency which the communication module found in the data base/lookup table and thus which the communication module expects.

As a result, if the particular regular module is not sending with the actual individual repetition frequency, an error signal may be outputted such as illuminating a warning lamp. However, also safety actions can be taken such as shutting down the particular regular module. Whether an error signal is enough or a further safety action is needed depends on the particular regular module. In case of an inverter module and/or in case of a socket module, safety actions will be taken in case of any error being found. If just a display module is having a malfunction, a warning signal might be enough.

According to one aspect, the individual repetition frequency is selected depending on the kind of regular module, in particular whether the regular module is in general involved in controlling energy of an electro surgical instrument connected to the surgical generator or not, and/or depending on an operating condition of the regular module, in particular, whether the regular module is in operation or in standby.

As explained above, there are regular modules which are more safety relevant than others. In particular, regular modules that are directly controlling the electro surgical instrument are considered to be safety relevant and thus a high individual repetition frequency should be selected. In other cases such as a display module, a small individual repetition frequency might be enough.

As also explained above, the individual repetition frequency can be low if the regular module is in standby or in any other mode that cannot lead to any harmful situation for a patient. Otherwise, a high repetition frequency should be selected.

The plurality of interconnected regular modules may comprise at least one further module as explained below.

A display module for displaying information for a user may be one interconnected regular module. Such display displays in particular operational situation of the electro surgical generator and could also indicate whether the electro surgical generator is in operation at all. It could also indicate a standby situation. Further features of and for such display module have been explained above.

Another interconnected regular module could be an input module for receiving commands from a user. Such input module could be a touchscreen, a switch and/or just a knob, for example, for switching the electro surgical generator on or off.

A further interconnected regular module could be at least a second inverter module for generating a feed signal for providing a high frequency energy for at least one electro surgical instrument with a different signal frequency than the first inverter module. This way, different electro surgical instruments can be operated in particular one after another, or also in parallel.

According to one aspect, the regular modules and the communication module are interconnected using a bus system, in particular a serial and/or differential bus system. This way, also the data frames sent with the repetition frequency can be sent via such bus system. Using a serial and/or differential bus system both provides a good resistance against electro smog which may be generated by any of the inverter modules. This also increases the safety of the system.

According to one aspect, each regular module sends a data frame comprising at least an identification code identifying the operational module, and an operation code, characterizing the operating state of the module. Optionally, the data frame may comprise additional information including information of boot up, error information, information on standby and/or information on a busy status.

It was found that such simple data frame is enough in order to check whether all these regular modules are functioning properly and are not having any error. Using such simple frame makes sure that safety provisions can be met. Preferably, the data frame comprises one byte for the identification code and a further byte for the operation code.

According to one aspect an error signal is generated, if a regular module is in a mode in which it should frequently be sending a communication frame at its individual repetition frequency, but the regular module is deviating from sending a communication frame at its individual repetition frequency by a predetermined minimum tolerance frequency. Such regular mode could be a test mode or a reactivity test mode in which the communication is focused on testing whether the particular regular module is without any error or malfunction.

To check this there can be a predetermined minimum tolerance frequency, which can be in a range of 10% to 50% of the repetition frequency. If the regular module is deviating from sending a communication frame at this repetition frequency, an error is indicated. Such error is indicated if the repetition frequency deviates by the predetermined minimum tolerance frequency. However, often such deviation from the individual repetition frequency maybe not sending any response at all. However, in order to better program such check algorithm and in order to immediately identify such malfunction said minimum tolerance frequency can be used.

In addition or alternatively for checking the reactivity a test signal is sent to the regular module to be tested and an error is assumed if an answer of the regular module to the test signal takes longer than a response time. The response time may be defined by an inverse value of the repetition frequency of the tested regular module. The error is assumed if the answer takes longer than the response time by a minimum time lag. So in that case an error signal is also generated. This kind of testing is similar to the first possibility but it is directly comparing a time lag of a response and the minimum time lag considered may be the inverse value of the predetermined minimum tolerance frequency. The minimum time lag may be in the range of 10 to 30% of the inverse of the repetition frequency.

According to the invention there is also suggested an electro surgical generator for controlling electro surgical instruments connected to the electro surgical generator and the electro surgical generator comprises
a plurality of interconnected regular modules, the plurality of interconnected regular modules comprising
at least one socket module for connecting an electro surgical instrument, and
at least one first inverter module for generating a feed signal for providing a high frequency energy as a feed signal for at least one electro surgical instrument connected to the socket module,

wherein
each regular module is adapted for communicating at least with a communication module,
each regular module is adapted to send a communication frame at an individual repetition frequency and
the individual repetition frequency is varied depending on a state of the electro surgical generator.

Preferably, the electro surgical generator is adapted to execute a method according to any of the aspects mentioned above.

Accordingly, the electro surgical generator operates as explained above with respect to any aspects of the method for controlling such electro surgical generator. For executing the method by the electro surgical generator such method can be implemented on a control module of the electro surgical generator. Such control module may be the communication module. However, as many steps are executed on different modules in the electro surgical generator the corresponding steps can be implemented at the respective modules. Accordingly, each regular module of the electro surgical generator may have a control module, which could also be a software or a processor connected to the regular module, or a software implemented on such processor. The particular method steps can be implemented on said respective regular module or its control module or on the communication module.

According to one aspect the electro surgical generator further comprises the communication module for controlling a communication with the regular modules, and/or each regular module is adapted to frequently send a communication frame at its individual repetition frequency to the communication module and/or to another regular module.

Accordingly, the electro surgical generator comprises such communication module which can be used as a central element for controlling the communication with the regular modules, as explained above. Each regular module may have stored its individual repetition frequency and is this way adapted to frequently send a communication frame at its individual repetition frequency. Besides that such regular module is in particular adapted to communicate using a bus system and via this bus system the communication frame can be send at its individual repetition frequency.

According to one aspect the plurality of interconnected regular modules comprises at least one further module of the list defined by
an energy distribution module for controlling an energy supply from the at least one inverter module to the at least one socket module,
a display module for displaying information for a user,
an input module for receiving demands from the user, and
at least a second inverter module for generating a feed signal for providing a high frequency energy for at least one electro surgical instrument with a different signal frequency than the first inverter module.

In addition or alternatively, the electro surgical generator comprises a monitoring module for monitoring if the communication module operates properly.

Accordingly, all these regular modules mentioned can have their own repetition frequency but some may also have a similar repetition frequency. In particular, the display module and the input module may have a much smaller repetition frequency, in particular at least 5 to 10 times smaller than the repetition frequency of the first inverter module, the second inverter module and/or the socket module. The energy distribution module may also have a high repetition frequency similar to the repetition frequency of the first and/or second inverter module and/or the socket module.

The monitoring module may have a high repetition frequency, in particular as high as the first inverter module, the second inverter module and/or the socket module.

According to one aspect it is suggested that the regular modules and the communication module are interconnected using a bus system, in particular a serial and/or differential bus system. Accordingly, a communication network inside the electro surgical generator is built up using such bus system, in particular a CAN bus system. This makes the communication resistant to electro smog.

According to the invention it was realized that a constant heartbeat frequency is inflexible in view of the different requirements of the modules in particular of the regular modules. Accordingly, the following solution, also explained above, was found.

Each regular module sends an own heartbeat having an own repetition frequency. The repetition frequency of the modules can be different to each other. The repetition frequencies maybe submitted at a start-up phase by the communication module, which can also be denoted as a central module, for each single regular module. Alternatively, each regular module has its own repetition frequency and communicates this to the communication module, i.e. to the central module.

Such communication module thus supervises the other regular modules, if the heartbeat frequency, i.e. the individual repetition frequency corresponds to predetermined values. A second module, i.e. the monitoring module monitors the central module. The central module may advise other regular modules to send with the higher or lower frequency the heartbeat.

The invention will now be explained by way of example based on the attached Figures.

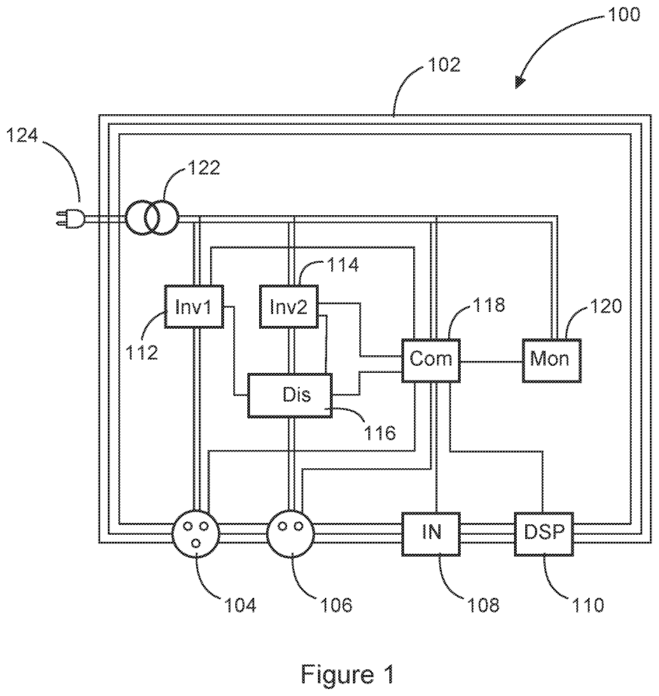

FIG. 1 shows an electro surgical generator in a schematic view.

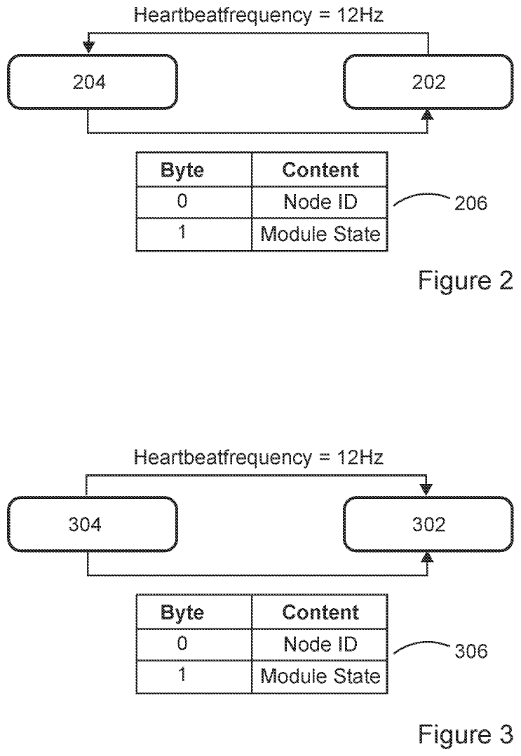

FIG. 2 illustrates one control concept of handling the repetition frequency.

FIG. 3 illustrates another control concept on how to handle the repetition frequency.

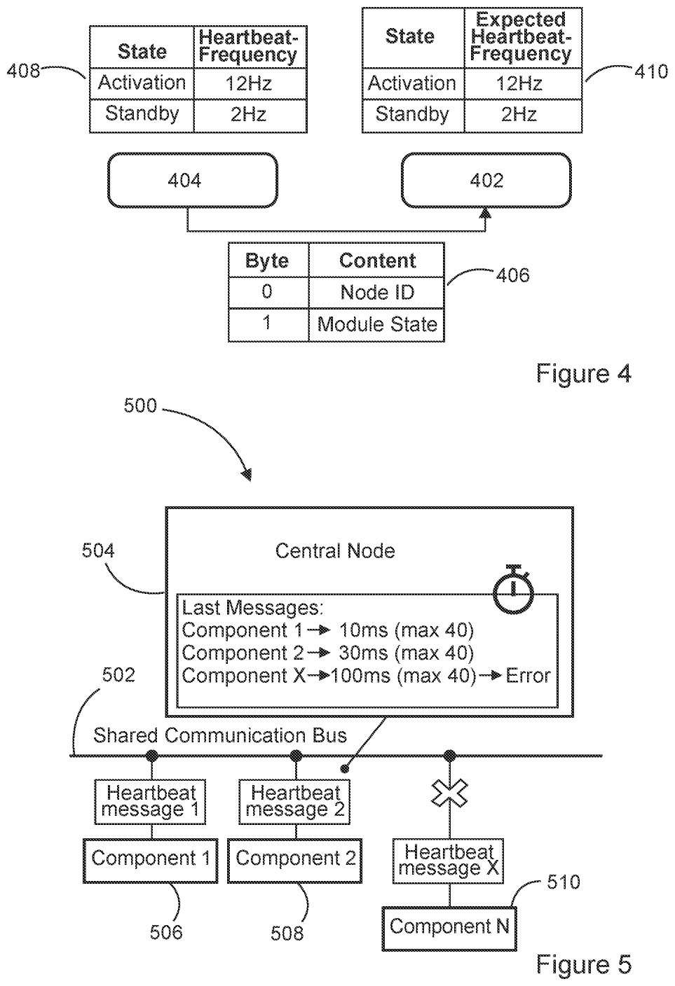

FIG. 4 illustrates a further control concept on how to handle the repetition frequency.

FIG. 5 illustrates a check routine and load situation of the electro surgical generator using a communication bus.

FIG. 1 shows in a schematic illustrative way an electro surgical generator 100 having a casing 102 illustrated by a triple line. In the casing 102 there may be mounted a first and second socket module 104 and 106 as well as an input module 108 and a display module 110. So these four modules could be attached at a front plate of the electro surgical generator 100 in the casing 102.

These four modules are regular modules. There are further regular modules, i.e. a first inverter module 112 and a second inverter module 114 as well as a distribution module 116. In addition, there is a communication module 118 and a monitoring module 120.

In addition, there is a power supply 122 illustrated as a transformer and having an electric plug 124.

For illustrative reasons double lines connecting modules or other components are used as a symbol for electric lines transmitting energy, whereas single lines are informational lines and can thus build communication network, in particular a bus system.

Accordingly, the power supply 122 provides electrical energy directly or indirectly to all modules. The input module 108 and the display module 110 may also get electric energy which is just not illustrated in order to not make the Figure to complex.

The first inverter module 112 may be an inverter module providing supersonic feed signal to a corresponding electro surgical instrument that can be plugged into the first socket module 104. The first inverter module 112 thus directly provides the first socket module 104 with energy, if in operation.

The second inverter module 114 may provide an electrical high frequency feed signal which is controlled and guided via the distribution module 116 to the second socket module 106. A different electro surgical instrument can be plugged into the second socket module 106.

For communication the communication module 118 is controlling each of the regular modules, i.e. it is controlling the first and second socket module 104 and 106, the input module 108, the display module 110, the first and second inverter module 112 and 114 and the distribution module 116. This is indicated by a single line connecting each single regular module with the communication module 118. Via these single lines which basically build the communication network and can be a serial bus system or part of it, such as a CAN bus. In addition, there is also a communication connection between the distribution module 116 and the first and second inverter 112 and 114. However, this communication line might not be necessary and any communication between these modules could also go via the communication module 118.

Accordingly, all of these regular modules have a kind of intelligence, i.e. a control module which can be implemented on a processor on each of these regular modules. In this way all of these regular modules are also capable of sending signals indicating their status, in particular if there is an error or not and also if they are in operation or in standby.

For indicating that a regular module is not having an error, there is a heartbeat signal sent from each regular module to the communication module 118. Such heartbeat signal can be a data frame comprising a node ID, namely an identification code identifying the particular regular module and a module state. The module state might just comprise an okay or a not okay. It could also have an information on whether the particular regular module is in standby or in operation and possibly in case there is an error, such error code could also be provided.

However, such data frame is sent with a repetition frequency from each regular module to the communication module 118. Each regular module may have an individual repetition frequency and thus with respect to another regular module a different repetition frequency. In particular, the repetition frequency can be 2 Hz or 12 Hz. Of course, other values could be used as well and possibly there could also be more than two different repetition frequencies.

In addition, there is the monitoring module 120, which is just monitoring the communication module 118. The communication module 118 checks for all the regular modules whether they are operating properly or are not having an error, i.e. if they send a so-called heartbeat signal with the repetition frequency and of course providing an information that everything is okay. This way, all regular modules are checked whether they were properly.

Only the communication module as does not check itself, as the electro surgical generator 100 has to fulfil a high safety requirement. Accordingly, such checking whether the regular modules work properly is necessary and in addition a redundancy should also be given such that this monitoring module 120 checks whether the communication module 118 is working properly. This can be also done in a similar way as for the regular modules, i.e. the communication module 118 may send a heartbeat signal to the monitoring module 120. The monitoring module then checks whether this heartbeat signal is received with the expected repetition frequency. The repetition frequency of the monitoring module maybe 10 Hz or more, in particular 12 Hz.

FIG. 2 illustrates a control concept on how to handle the repetition frequency. This diagram illustrates a supervising node 202 and a CAN-node 204. The supervising node 202 represents the communication module 118, or a corresponding node of the communication module 118. The CAN-node 204 indicates that a CAN bus system is used and the CAN-node represents any regular module. I.e. could be any regular module of the regular modules explained with respect to FIG. 1, and also other regular modules.

FIG. 2 thus illustrates that the supervising node 202, thus the communication module 118 sends a repetition frequency, which can also be denominated as heartbeat frequency, as a set value to the CAN-node 204 and thus to one regular module. The value illustrated in FIG. 2 is 12 Hz but it could also be another value and it could also be varying between the regular modules to which this value is sent.

Based on that repetition frequency received from the supervising node the CAN-node 204 sends with this repetition frequency a data frame to the supervising node 202. Such data frame is illustrated by the table 206 shown in FIG. 2.

Accordingly, this data frame has a node ID and thus an identification number of the particular CAN-node, thus of the regular module, and has a module state. The module state could just be an okay or an error, or it could be an indication whether the particular regular module is in operation or in standby.

Accordingly, said data frame illustrated by table 206 is sent from the CAN-node 204, thus sent from a regular module to the supervising node 202, thus to the communication module, which could be the communication module 118 according to FIG. 1. If this data frame is sent with the repetition frequency and also providing at least the status okay, the supervising node 202 identifies that there is no error in the particular regular module.

FIG. 3 is similar to FIG. 2 and shows also a supervising node 302 and a CAN-node 304. These can basically be the same as the supervising node 202 and the CAN-node 204 according to FIG. 2. Basically, the only difference in this control concept shown in FIG. 3 is that the CAN-node 304 and thus the regular module which this CAN-node 304 represents, already has its repetition frequency and sends this repetition frequency to the supervising node 302. Accordingly, the supervising node 302 knows which repetition frequency to expect.

Based on that the concept works as shown in FIG. 2, i.e. the CAN-node 304 sends a data frame with the repetition frequency and the data frame is illustrated by the table 306.

Accordingly, the supervising node 304 has no control of the heartbeat frequency. The node itself, i.e. the regular module reports in which frequency the heartbeat frame is sent. This has the advantage that there is a module specific handling and risks. For example, a temperature sent does not need to be supervised very fast because failures would not immediately result in critical situations. There is also a modular approach possible. That means independent development of each module with their own specification is possible and this enables an exchangeability of such modules. However, there could be an advantage that no central control of an over bus load is possible.

Another control concept of handling the repetition frequency is illustrated in FIG. 4. FIG. 4 also has a supervising node 402 and an CAN-node 404.

According to this concept, neither the supervising node sends a repetition frequency to the CAN-node nor sends the CAN-node its repetition frequency to the supervising node. Instead the repetition frequency depends on a state of the electro surgical generator, i.e. whether it is in activation or in standby. These different states have different repetition frequencies assigned to. In the given example there is 12 Hz for the activation and 2 Hz for the standby. This is illustrated in the first lookup table 408.

Based on that the CAN-node 404 sends a data frame with the repetition frequency to the supervising node 402. The general function of these two nodes and what they are representative for is explained in FIGS. 2 and 3. Accordingly, the CAN-node 404 is representative for any regular module and the supervising node 402 is representative for the communication module. The data frame sent over is also illustrated by the table 406 which has been explained with respect to table 206 in FIG. 2.

However the used repetition frequency depends on the first lookup table 408 and accordingly said data frame represented by the table 406 is sent with the repetition frequency of 2 Hz if the generator is in standby and with the repetition frequency of 12 Hz if the generator is in an activation state.

The supervising node 402 is having a second lookup table 410. In this second lookup table 410 the expected heartbeat frequency and thus the expected repetition frequency is recorded. The expected heartbeat frequency according to the second lookup table 410 is thus corresponding to the given heartbeat frequency according to the first lookup table 408.

Therefore, the supervising node 402 checks if the CAN-node 404 sends the data frame according to table 406 with the correct repetition frequency, i.e. the correct heartbeat frequency according to its second lookup table 410.

To summarize FIG. 4, the modules have a lookup table for heartbeat frequencies per module. There are two variants:
The lookup table is fixed between all modules. Accordingly, It is a fixed and specified table which is the same on all nodes. All operating modes are globally the same. In particular the mode or status can be activated and standby.
The lookup table is communicated for example during boot up with module specific parameters.

This table is the link between the heartbeat frequency and the operational mode of the module. The operational mode might be:
Based on global system state for example generator is in activation mode or standby.
Module dependent, e.g. instrument is currently not connected, so the repetition frequency, i.e. the heartbeat frequency can be reduced.

Advantages are as explained with respect to FIGS. 2 and 3. In addition, a very granular selection of surveillance time can be programmed. There is also a modular approach possible. I.e. there is possible an independent development of each module with their own specification enabling an exchangeability. There might be a small disadvantage such that the lookup table needs memory and management by surveillance node and it might be a more complex implementation with respect to the other possibilities explained above.

FIG. 5 illustrates a check routine and load situation of the electro surgical generator using a communication bus. It shows a bus system 500 having a shared communication bus, i.e. a bus 502 to which a communication module 504 and three regular modules 506, 508 and 510 are connected. The communication and the illustrated checking whether any of the regular modules is without any malfunction is coordinated by the communication module 504. The communication module 504 can also be denominated as central node. This communication module frequently receives information signals from these three regular modules and of course from further modules as well, if any. In this illustration it is assumed that the communication module expects every regular module to respond or send data frames within an repetition time of 40 ms. Accordingly, it checks whether it receives messages from each regular module 506, 508 and 510 within this repetition time of 40 ms.

Based on that, there is a situation assumed that the first regular module 506 sends its message within 10 ms after its last message. Accordingly, this 10 ms is within the maximum repetition time of 40 ms.

The second regular module 508 sends its message within 30 ms after the last message and that is also within the limit of 40 ms for the repetition time.

However, the third regular module which may also be representative for any further regular modules, is sending its message after 100 ms. Accordingly, these 100 ms are bigger than the repetition time of 40 ms which is thus a maximum repetition time of 40 ms. Accordingly, for the third regular module 510 an error was identified and accordingly any error signal or safety actions will take place in the next step.

As a background of the invention it was also found that in a distributed system with multiple hardware and software components that communicate to each other, the overall system integrity must be ensured. If critical components fail, the system security or critical functions cannot be assured. Therefore, every component of a generator may incorporate mechanisms to supervise that every software and hardware component is working properly and reacts with a given output.

In principle a super-vising system receives a message, that could be a heartbeat, hardware signal or digital message, from a component, i.e. a regular module, on a regular basis. If the message is not received in a given/specified time frame, that could be monitored using a timeout, the supervisor expects that the system is stalled and has to incorporate further actions to return to a stable system state. This can be a system reset or error handling.

In the current system the software components (called “tasks”) are using this system. Every task sends a message to the system supervisor, which could be implemented in the communication module, and could be denominated as task supervisor. These messages could be sent at least every 60 ms. If a task is not sending a message in the given timeframe, the system is rebooted. An external Watchdog circuit supervises the task supervisor and the CPU. If the task supervisor does not reset the external watchdog, a hardware reset of the CPU could be triggered.

1. A method for controlling an electro surgical generator for controlling electro surgical instruments connected to the electro surgical generator, comprising
a plurality of interconnected regular modules, the plurality of interconnected regular modules comprising
at least one socket module for connecting the electro surgical instrument, and
at least one first inverter module for generating a feed signal for providing a high frequency energy for at least one electro surgical instrument connected to the socket module, wherein,
each regular module communicates at least with another regular module and/or with a communication module,
each regular module is sending a communication frame at an individual repetition frequency and
the individual repetition frequency is varied depending on a state of the electro surgical generator.

2. The method according to claim 1, wherein
the electro surgical generator further comprises the communication module for controlling a communication with the regular modules, and/or
each regular module sends a communication frame at its individual repetition frequency to the communication module and/or to another regular module, and/or
the electro surgical generator and/or each regular module perform a self-check during standby, wherein each regular module sends a communication frame at its individual repetition frequency.

3. The method according to claim 1, wherein
at least during a standby mode the individual repetition frequencies of two, more than two or all of the other regular modules are the same.

4. The method according to claim 1, wherein
some or all individual repetition frequencies are selected depending on an overall state of the electro surgical generator and/or
one, some or all of the individual repetition frequencies are selected depending on a state of the corresponding regular module.

5. The method according to claim 1, wherein
during standby of the electro surgical generator some or all individual repetition frequencies are selected to be lower when compared to the electro surgical generator not being in standby
during standby of the electro surgical generator an individual repetition frequency is selected being smaller than the activation frequency, and
when the electro surgical generator is not in standby, an individual repetition frequency is selected being larger than the activation frequency.

6. The method according to claim 1, wherein
during activation of the electro surgical generator as to leave a standby mode,
the communication module sends a command to one, some or all of the regular modules to increase their individual repetition frequency and/or
when the electro surgical generator enters the standby mode
the communication module sends a command to one, some or all of the regular modules to decrease their individual repetition frequency.

7. The method according to claim 1, wherein
one, some or all of the regular modules each individually alter their individual repetition frequency depending on their module state.

8. The method according to claim 1, wherein
the communication module frequently checks the individual repetition frequencies of the regular modules depending on
an individual module state of the corresponding module, and/or
depending on an overall state of the electro surgical generator, and/or
based on a given data base which is particularly stored in a memory of the communication module, and wherein
the communication module provides an error signal and optionally initiates safety actions if at least one of the checked individual repetition frequencies deviates from the corresponding expected individual repetition frequency.

9. The method according to claim 1, wherein
the individual repetition frequency is selected
depending on the kind of regular module, and/or
depending on an operating condition of the regular module.

10. The method according to claim 1, wherein
the plurality of interconnected regular modules comprises at least on further module of the list defined by
a display module for displaying information for a user,
an input module for receiving demands from the user,
at least a second inverter module for generating a feed signal for providing a high frequency energy for at least one electro surgical instrument with a different signal frequency than the first inverter module.

11. The method according to claim 1, wherein
the regular modules and the communication module are interconnected using a bus system.

12. The method according claim 1, wherein
each regular module sends a data frame comprising at least
an identification code identifying the operational module, and
an operation code, characterizing the operating state of the module, and optionally
additional information including information of boot-up, error information, information on standby and/or information on a busy-status.

13. The method according to claim 1, wherein
an error-signal is generated if
a regular module is in a mode of frequently sending a communication frame at its individual repetition frequency and
the regular module is deviating from sending a communication frame at its individual repetition frequency by a predetermined minimum tolerance frequency, and/or if
for checking a reactivity a test signal is sent to the regular module to be tested, and
an answer of the regular module to the test signal is taking longer than a time defined by an inverse value of the repetition frequency of the tested regular module by a minimum time lag.

14. An electro surgical generator for controlling electro surgical instruments connected to the electro surgical generator, comprising
a plurality of interconnected regular modules, the plurality of interconnected regular modules comprising
at least one socket module for connecting an electro surgical instrument, and
at least one first inverter module for generating a feed signal for providing a high frequency energy as a feed signal for at least one electro surgical instrument connected to the socket module, wherein
each regular module is adapted for communicating at least with a communication module,
each regular module is adapted to send a communication frame at an individual repetition frequency and
the individual repetition frequency is varied depending on a state of the electro surgical generator, and
the electro surgical generator is adapted to execute a method according to claim 1.

15. The electro surgical generator according to claim 14, wherein
the electro surgical generator further comprises the communication module for controlling a communication with the regular modules, and/or
each regular module is adapted to frequently send a communication frame at its individual repetition frequency to the communication module and/or to another regular module.

16. The electro surgical generator according to claim 14, wherein
the plurality of interconnected regular modules comprises at least one further module of the list defined by
an energy distribution module for controlling an energy supply from the at least one inverter module to the at least one socket module,
a display module for displaying information for a user,
an input module for receiving demands from the user, and
at least a second inverter module for generating a feed signal for providing a high frequency energy for at least one electro surgical instrument with a different signal frequency than the first inverter module and/or
the electro surgical generator comprises a monitoring module for monitoring if the communication module operates properly.

17. The electro surgical generator according to claim 14, wherein
the regular modules and the communication module are interconnected using a bus system.
